(19) United States
(12) Patent Application Publication
(10) Pub. No.: US 2008/0199537 A1
(43) Pub. Date: Aug. 21, 2008

(54) TOPICAL GATIFLOXACIN FORMULATIONS

(75) Inventors: Haresh G. Bhagat, Fort Worth, TX (US); Ramon L. Espino, Cleburne, TX (US)

Correspondence Address: ALCON, IP LEGAL, TB4-8, 6201 SOUTH FREEWAY, FORT WORTH, TX, 76134, US

(73) Assignee: ALCON RESEARCH, LTD., Fort Worth, TX (US)

(21) Appl. No.: 12/031,772

(22) Filed: Feb. 15, 2008

Related U.S. Application Data
(60) Provisional application No. 60/890,543, filed on Feb. 19, 2007.

(51) Int. Cl. A61K 33/22; A61P 27/02

(52) U.S. Cl. 424/659

(57) ABSTRACT

Topical ophthalmic solution compositions of gatifloxacin are disclosed. The compositions do not contain disodium edetate or any other metal chelating agent.

This application claims priority to U.S. Provisional Application, U.S. Ser. No. 60/890,543, filed on Feb. 19, 2007.

### BACKGROUND OF THE INVENTION

This invention relates to topically administrable ophthalmic formulations of gatifloxacin.

Gatifloxacin is a known compound. See U.S. Pat. No. 4,980,470. It is commercially available as Zymar® (gatifloxacin ophthalmic solution) 0.3% from Allergan, Inc. According to its package insert, this product contains disodium edetate as an inactive ingredient. U.S. Pat. No. 6,333,045 discloses aqueous liquid pharmaceutical preparations of gatifloxacin or its salts and disodium edetate. According to the '045 patent, disodium edetate increases the corneal permeability of gatifloxacin, increases the solubility of gatifloxacin at about physiological pH, and can prevent coloration of aqueous liquid preparations of gatifloxacin

### SUMMARY OF THE INVENTION

The compositions of the present invention are aqueous solution compositions of gatifloxacin. The compositions consist essentially of gatifloxacin or a pharmaceutically acceptable salt thereof, polyquaternium-1, boric acid, propylene glycol, and water, and optionally contain an ophthalmically acceptable pH-adjusting agent if necessary to adjust pH to 5.8-6.2. The compositions do not contain disodium edetate, yet they are sufficiently stable to provide a commercially desirable shelf-life.

### DETAILED DESCRIPTION OF THE INVENTION

Unless indicated otherwise, all ingredient concentrations are presented in units of % weight/volume (% w/v).

The compositions of the present invention consist essentially of gatifloxacin or a pharmaceutically acceptable salt thereof, polyquaternium-1, boric acid, propylene glycol, and water, and optionally contain an ophthalmically acceptable pH-adjusting agent.

Gatifloxacin is available in a variety of forms. See, for example, U.S. Pat. No. 6,413,969 (gatifloxacin pentahydrate), U.S. Pat. No. 5,043,450 (gatifloxacin hemihydrate), U.S. Pat. No. 5,880,283 (gatifloxacin sesquihydrate), U.S. Patent Application Publication No. 2004/0009989 (crystalline forms “A,” “B,” “C,” “D,” “El,” “F,” “G,” “H,” “I,” and “J” of gatifloxacin) and U.S. Patent Application Publication No. 2004/0038988 (crystalline forms “O,” and “V” of gatifloxacin). Yet another form of gatifloxacin is described in WO 2005/047262. The compositions of the present invention contain gatifloxacin in an amount of 0.25-0.55% (w/v), preferably 0.3% (w/v) or 0.5% (w/v), and most preferably 0.3% (w/v).

In addition to gatifloxacin, the aqueous compositions of the present invention contain polyquaternium-1. Polyquaternium-1, also known as POLYQUAD® preservative, is a known preservative agent for topical ophthalmic compositions. See, for example, U.S. Pat. No. 5,603,929. The polyquaternium-1 preferably has a number average molecular weight from 2,000 to 30,000, and more preferably from 3,000 to 14,000. The compositions of the present invention contain polyquaternium-1 in an amount of 0.0005-0.0015% (w/v), preferably 0.001% (w/v).

The compositions of the present invention contain boric acid in an amount from 0.4-0.8% (w/v), preferably 0.6% (w/v).

Propylene glycol acts as a nonionic tonicity-adjusting agent and a stabilizing agent. It is known for use in topical ophthalmic compositions. It is contained in the compositions of the present invention in an amount sufficient to cause the compositions to have an osmolality from 260-330 mOsm/kg, preferably about 280-300 mOsm/kg, and most preferably about 285-300 mOsm/kg. In one embodiment, the compositions of the present invention contain propylene glycol in an amount from 1-1.5% (w/v), preferably 1.3-1.4%, and most preferably 1.35%.

The pH of the aqueous compositions of the present invention is adjusted with an ophthalmically acceptable pH-adjusting agent. Ophthalmically acceptable pH adjusting agents are known and include, but are not limited to, hydrochloric acid (HCl) and sodium hydroxide (NaOH). The compositions of the present invention preferably contain NaOH or HCl to obtain a composition pH of 5.8-7.0. Most preferred is a composition pH of 6.0.

The following examples are intended to illustrate, but not limit, the present invention.

### EXAMPLE 1

Topical Ophthalmic Solution

| Ingredient | Formulation A (% w/v) |
|---|---|
| Gatifloxacin | 0.25-0.55 |
| Polyquaternium-1 | 0.0005-0.0015 |
| Boric Acid | 0.4-0.8 |
| Propylene Glycol | 1-1.5 |
| NaOH/HCl | q.s. pH 5.8-7.0 |
| Purified Water | q.s. 100 |

### EXAMPLE 2

Preferred Topical Ophthalmic Solution

| Ingredient | Formulation B (% w/v) |
|---|---|
| Gatifloxacin | 0.3 |
| Polyquaternium-1 | 0.001 |
| Boric Acid | 0.6 |
| Propylene Glycol | 1.35 |
| NaOH/HCl | q.s. pH 6.0 |
| Purified Water | q.s. 100 |

### EXAMPLE 3

Preferred Topical Ophthalmic Solution

| Ingredient | Formulation B (% w/v) |
|---|---|
| Gatifloxacin | 0.5 |
| Polyquaternium-1 | 0.001 |
| Boric Acid | 0.6 |
| Propylene Glycol | 1.35 |
| NaOH/HCl | q.s. pH 6.0 |
| Purified Water | q.s. 100 |

### EXAMPLE 4

### Preservative Efficacy Test Results

Three lots of the formulation of Example 2 were prepared and subjected to preservative efficacy testing. Antimicrobial preservative effectiveness was determined using an organism challenge test according to the methods described in the United States Pharmacopeia (USP) and European Pharmacopoeia (Ph.Eur.). Samples were inoculated with known levels of one or more of the following: gram-positive (Staphylococcus aureus ATCC 6538) and gram-negative (Pseudomonas aeruginosa ATCC 9027 and Escherichia coli ATCC 8739) vegetative bacteria, yeast (Candida albicans ATCC 10231) and mold (Aspergillus niger ATCC 16404). The samples were then pulled at specified intervals to determine if the antimicrobial preservative system was capable of killing or inhibiting the propagation of organisms purposely introduced into the formulation. The rate or level of antimicrobial activity determines compliance with the USP and/or Ph.Eur. preservative efficacy standards for ophthalmic preparations.

The compendial preservative standards for ophthalmic preparations are presented below:

| | Log Reduction of | | |
|---|---|---|---|
| | Organism Population | | |
| | | | Ph.Eur. B |
| Time Pull | USP | Ph.Eur. A | (Min) |

For Bacteria:

| | | | |
|---|---|---|---|
| 6 hours | — | 2 | — |
| 24 hours | — | 3 | 1 |
| 7 days | — | — | 3 |
| 14 days | 3 | — | — |
| 28 days | NI | NR | NI |

For Fungi:

| | | | |
|---|---|---|---|
| 7 days | — | 2 | — |
| 14 days | NI | — | 1 |
| 28 days | NI | NI | NI |

NR = No organisms recovered
NI = No increase at this or any following time pulls
— = No requirement at this time pull

The results of the microorganism challenge tests are shown in Tables 1 and 2 below.

TABLE 1

Preservative
Efficacy
Standard

| | | Ph.Eur. B |
|---|---|---|
| Formulation of Example 2 | USP | (Minimum) |
| Lot 1 | Pass | Pass |
| Lot 2 | Pass | Pass |
| Lot 3 | Pass | Pass |

TABLE 2

| | | | | | | |
|---|---|---|---|---|---|---|
| Lot # | Log. Int. | 6 hours | 24 hours | 7 days | 14 days | 28 days |

S. aureus

| | | | | | | |
|---|---|---|---|---|---|---|
| 1 | 5.9 | 4.9 | 4.9 | 4.9 | 4.9 | 4.9 |
| 2 | 5.9 | 4.9 | 4.9 | 4.9 | 4.9 | 4.9 |
| 3 | 5.9 | 4.9 | 4.9 | 4.9 | 4.9 | 4.9 |

P. Aeruginosa

| | | | | | | |
|---|---|---|---|---|---|---|
| 1 | 6.0 | 5.0 | 5.0 | 5.0 | 5.0 | 5.0 |
| 2 | 6.0 | 5.0 | 5.0 | 5.0 | 5.0 | 5.0 |
| 3 | 6.0 | 5.0 | 5.0 | 5.0 | 5.0 | 5.0 |

E. Coli

| | | | | | | |
|---|---|---|---|---|---|---|
| 1 | 6.0 | 5.0 | 5.0 | 5.0 | 5.0 | 5.0 |
| 2 | 6.0 | 5.0 | 5.0 | 5.0 | 5.0 | 5.0 |
| 3 | 6.0 | 5.0 | 5.0 | 5.0 | 5.0 | 5.0 |

C. Albicans

| | | | | | | |
|---|---|---|---|---|---|---|
| 1 | 6.1 | — | — | 5.1 | 5.1 | 5.1 |
| 2 | 6.1 | — | — | 5.1 | 5.1 | 5.1 |
| 3 | 6.1 | — | — | 5.1 | 5.1 | 5.1 |

A. Niger

| | | | | | | |
|---|---|---|---|---|---|---|
| 1 | 6.1 | — | — | 1.1 | 1.2 | 1.9 |
| 2 | 6.1 | — | — | 1.2 | 1.7 | 1.8 |
| 3 | 6.1 | — | — | 1.2 | 1.8 | 1.7 |

The invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims rather than by the foregoing description.

What is claimed is:

1. A topically administrable ophthalmic composition consisting essentially of
a) 0.25-0.55% (w/v) gatifloxacin or a pharmaceutically acceptable salt thereof;
b) 0.0005-0.0015% (w/v) polyquaternium-1;
c) 0.4-0.8% boric acid
d) 1.0-1.5% (w/v) propylene glycol;
e) an ophthalmically acceptable pH-adjusting agent in an amount sufficient to cause the composition to have a pH from 5.8-7.0; and
f) water.

2. A topically administrable ophthalmic composition consisting essentially of
a) 0.3% (w/v) gatifloxacin;
b) 0.001% (w/v) polyquaternium-1;
c) 0.6% boric acid
d) 1.35% (w/v) propylene glycol;
e) NaOH or HCl in an amount sufficient to cause the composition to have a pH from 5.8-6.2; and
f) water.

3. A topically administrable ophthalmic composition consisting essentially of
a) 0.5% (w/v) gatifloxacin;
b) 0.001% (w/v) polyquaternium-1;
c) 0.6% boric acid
d) 1.35% (w/v) propylene glycol;
e) NaOH or HCl in an amount sufficient to cause the composition to have a pH from 5.8-6.2; and
f) water.
